(19) United States
(12) Patent Application Publication
(10) Pub. No.: US 2009/0011969 A1
(43) Pub. Date: Jan. 8, 2009

(54) Method for Cleaning Metal Parts

(76) Inventor: Frank-Peter Lang, Hattersheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 12/223,568

(22) PCT Filed: Feb. 3, 2007
(86) PCT No.: PCT/EP2007/000930

§ 371 Date: Jul. 31, 2008

(30) Foreign Application Priority Data
Feb. 8, 2006 (DE) 10 2006 006 022.9

(51) Int. Cl. C23G 5/032; C07C 43/30; C23G 1/00

(52) U.S. Cl. 510/258; 568/600; 510/245; 568/592

(57) ABSTRACT

The invention relates to a method for cleaning metal parts, according to which the metal parts are treated with a compound of the formula (1)

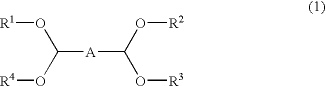

wherein A represents (CH₂)ₐ or phenylene and R¹, R², R³ and R⁴ are identical or independently of one another represent C₁ to C₆-n-alkyl and/or isoalkyl and a represents a whole number from 0 to 4.

The invention relates to a novel class of solvents and mixtures thereof with further components for cleaning metals.

Metals are valuable materials for the production of capital goods as well as consumer goods. Iron, steel, zinc and galvanized steel, chrome-plated steel, nickel-plated steel, stainless steel, copper, brass, aluminum, tin, titanium, magnesium and a very wide range of metal alloys are used for this purpose. Metals are used in machine and plant construction, in automotive construction, for railroad vehicles, in the aircraft industry, in ship building, in the construction industry, for household appliances and in many further areas.

The metallic parts which have to be cleaned for further processing and use are, for example, parts turned on automatic lathes, machine components, hydraulic valves, lapped parts of high-pressure pumps, pneumatic parts, diecast parts, lock cylinders, tools, gear wheels, fittings, polished parts, instrument sets, precision engineering parts, loom parts, aircraft components, car bodyworks, cold-worked parts and precision punched parts, metal supports, metal plates, metal struts, etc.

Various metal processing auxiliaries are used for processing the metal parts, such as, for example, cooling lubricants, drawing agents, polishing and lapping pastes, preservatives, corrosion inhibitors, abrasives, fluxes, release agents, pickles, cutting oils, drilling oils and further auxiliaries, are used for processing the metal parts. After the mechanical processing of the workpiece, its surface must be thoroughly cleaned for further processing steps. The contaminants adhering to the metal surfaces are firstly residues of the abovementioned metal processing auxiliaries and secondly particulate dirt due to, for example, metal turnings or dust. The metal processing auxiliaries contain formulation constituents which are strongly adsorbed on the surface of the metal, which is a precondition for their effectiveness. However, these interfere with subsequent processes, such as a thermochemical treatment, or—in the case of aqueous surface cleaning—may react with the detergent ingredients to give components forming a contamination layer. However, the removal of all impurities is an important precondition for the further error-free processing of metal parts by, for example, thermochemical processes (gas nitriding, nitrocarburization, etc.), electrochemical processes, coating, phosphating, galvanizing, chrome-plating, nickel-plating, lacquering, soldering, welding, etc.

For removing the impurities adhering to the metal surfaces, the following cleaning agents and cleaning methods were or are used.

Chlorofluorocarbons (CFCs) have an excellent dissolving power for fatty and oily surface dirt and are non-toxic. However, since they are suspected of being mainly responsible for the degradation of the ozone layer in the Earth's atmosphere, they are no longer permitted in many countries.

Chlorinated hydrocarbons (CHCs), such as, for example, perchloroethylene (PER), exhibit an outstanding dissolving power for oils and fats, comparable with the CFCs. Owing to their toxicological properties, however, they must be used in completely closed plants. Thus, for example, the disadvantages of PER are in particular its potential carcinogenic effect in humans, its ready solubility in fat-containing foods and its considerable water hazard properties.

PER is classified as a “dangerous substance” in the “black list” of the EU and as a hazardous substance in the meaning of the regulation on hazardous substances. Other CHCs, such as trichloroethene, 1,1,1-trichloroethane and dichloromethane, are also toxicologically unsafe.

Cold cleaners based on halogen-free hydrocarbons (hydrocarbon solvents, HS) have, like the chlorofluorocarbons (CFCs) and the chlorinated hydrocarbons (CHCs), a very good cleaning effect with respect to hydrophobic surface dirt. However, in order also to have a good action against pigment dirt, water-soluble organic compounds or inorganic salts, they must be used in aqueous emulsion which is produced, for example, by means of ultrasound or by injection flooding. Fractions having a boiling range from about 180° C. to about 330° C., preferably from 180° C. to 240° C., are used as hydrocarbons. Since the boiling range is very much higher than the boiling points of the CFCs and of the CHCs, the final drying process is substantially more time-consuming and energy-consuming.

Semi-aqueous cleaning methods combine cleaning with a cleaning agent based on organic solvents with an aqueous washing operation.

Aqueous cleaners (surfactant cleaners) substantially contain inorganic builders (such as alkali metal hydroxides, silicates, phosphates, borax, sodium carbonate), complexing agents (such as gluconates, phosphonates), surfactants (anionic and/or nonionic) and corrosion inhibitors (fatty acids and ethanolamines). Against adhering fats and oils, they have no cleaning power comparable with that of the readily volatile organic solvents. However, compared with the organic solvents, they have an improved cleaning power with respect to pigment dirt (chips, abraded material, graphite). Another disadvantage of the use of aqueous cleaners is that components thereof can be removed only by thorough rinsing with pure water. Cleaning agent residues themselves constitute contamination on the cleaned metal surface. A further disadvantage is that plants for these cleaners require a more complicated plant technology.

There is thus still the need for cleaning agents which combine a very good cleaning power with an advantageous toxicological or ecological rating and which, owing to their physicochemical properties, are to be rated as being better than the prior art. They should moreover be capable of being used substantially in the cleaning methods which are used in accordance with the prior art for cleaning metal parts.

Methods for degreasing metals are, for example, cold cleaning, steam degreasing, immersion, spraying, irrigation, cleaning by means of ultrasound and processes in which various methods are combined with one another. Furthermore, manual methods comprising lapping, cleaning with a paint brush or brushing may be used. A distinction is furthermore made between one-stage and multi-stage cleaning units. Halogenated hydrocarbons (HHCs) are used as a rule in simple, one-stage units. On the other hand, the aqueous cleaners (surfactant cleaners) have to be used in complicated, multi-stage units owing to the different cleaning mechanism and the complex cleaner composition and for ensuring freedom from spots. These consist as a rule of a coarse cleaning stage, a fine cleaning stage and a plurality of washing stages for removing cleaning agent residues.

The object of the present invention is to provide cleaning agents which better meet the abovementioned requirements for chemical cleaning than the cleaners used according to the prior art and which have a good toxicological and ecological property profile.

It has somewhat surprisingly been found that compounds of the formula (1) have a better cleaning power or dissolving power for removing fats and oils from metallic surfaces than the organic solvents used according to the prior art. They are also to be rated toxicologically and ecologically as being substantially more advantageous than chlorinated hydrocarbons or halogen-free hydrocarbons. Furthermore, like aqueous, surfactant-containing cleaning agents, they have a good dirt dispersing power. They are therefore excellently suitable as cleaning agents for metal cleaning.

The invention therefore relates to a method for cleaning metal parts, which comprises treating the metal parts with a compound of the formula (1)

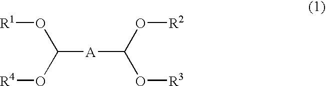

in which

A is (CH₂)ₐ or phenylene and R¹, R², R³ and R⁴ are identical or, independently of one another, are C₁ to C₆-n-alkyl and/or isoalkyl and a is an integer from 0 to 4.

Preferably, R¹, R², R³ and R⁴ are identical or, independently of one another, are C₁ to C₄-n-alkyl and/or isoalkyl and a is 0.

Very particularly preferably, R¹, R², R³ and R⁴ are identical or, independently of one another, are C₁- and/or C₂₋alkyl and a is 0.

Examples of the radicals R¹ to R⁴ are, for example: methyl-, ethyl-, n-propyl-, isopropyl-, n-butyl-, isobutyl-, sec-butyl-, tert-butyl-.

The compounds of the formula (1) are acetals. Acetals are generally obtained by the reaction of aldehydes with 2 mol of an alcohol per carbonyl group in the presence of catalysts, such as, for example, dry hydrogen chloride.

For the synthesis of compounds of the formula (1), it is necessary to use dialdehydes. Preferred dialdehydes for the synthesis of compounds of the formula (1) are glyoxal, malondialdehyde (1,3-propanedial, 1,3-propanedialdehyde), 1,4-butanedial or terephthalaldehyde.

A very preferred dialdehyde is glyoxal, which leads to compounds of the formula (1) with a=0.

Particularly preferred compounds for the intended use described are tetramethoxyethane and tetraethoxyethane.

Compounds of the formula (1) can be used in all the abovementioned methods for cleaning metals, the cleaning of untreated metal parts, i.e. those which are not lacquered, being preferred.

The method according to the present invention relates only to the cleaning of metal parts in the industrial and commercial sector. The cleaning of metals with oven cleaners, grill cleaners, stainless steel cleaners, wheel rim cleaners and engine cleaners is therefore excluded. These types of use occur only in the area of household use.

The cleaning can be effected alone or in combination with other customary cleaning agent constituents, such as, for example, surfactants, builders and other organic solvents. The compounds of the formula (1) can be used by the following methods:
1) Cleaning exclusively with a compound of the formula (1). If appropriate, stabilizers or corrosion inhibitors can be added.
2) Cleaning with a compound of the formula (1) and with the addition of other organic solvents. Here too, stabilizers or corrosion inhibitors can, if appropriate, be added.
3) Cleaning with a compound of the formula (1) and other organic solvents, these being used in a separate cleaning step.
4) Cleaning with a compound of the formula (1), if appropriate with addition of up to 30%, preferably up to 20% and very particularly preferably up to 10% of water and with addition of up to 20%, preferably up to 10%, of surfactants, builders, complexing agents and corrosion inhibitors.
5) Cleaning methods in which first a cleaning step according to the abovementioned methods 1 to 3 and then an aqueous aftertreatment are effected.
6) Cleaning methods in which first a pure aqueous cleaning step and then an aftertreatment with a cleaning step according to the abovementioned methods 1 to 3 are effected. An aftertreatment with the compound of the formula (1) or its combination with other solvents can serve, for example, for faster and more energy-saving drying after the aqueous cleaning step.

If it is intended to combine a compound of the formula (1) with other detergent substances, these may be, for example:

Organic, water-soluble, partly water-soluble or water-insoluble solvents, such as:
Monohydric alcohols, such as ethanol, n-propanol, isopropanol, n-butanol, isobutanol and tert-butanol.
Di- or polyhydric alcohols, such as ethylene glycol, 1,2-propylene glycol, 1,3-propylene glycol, butylene glycol or glycerol.
Ethers, in particular glycol ethers, which are obtained by the reaction of C₁-C₆-alcohols or of phenol with one or more moles of an alkylene oxide, in particular with ethylene oxide or propylene oxide. Examples of glycol ethers are mono-, di- and tripropylene glycol monomethyl ether, propylene glycol phenyl ether, mono- and diethylene glycol n-butyl ether, ethylene glycol phenyl ether.
Ketones, such as, for example, methyl isopropyl ketone and 2-butanone.
Esters, e.g. propyl acetate.
Oligo- and polyalkylene glycols, such as, for example, diethylene glycol, dibutylene glycol or low molecular weight polyethylene glycol, for example having a molar mass of 300 and 400 (PEG 300 and PEG 400).
n-Alkanes and isoalkanes (hydrocarbons) of different chain lengths, with different degree of branching or with specific boiling ranges.
Nitrogen-containing solvents, such as, for example, N-methylpyrrolidone.
Chlorinated hydrocarbons, such as methylene chloride, 1,1,1-trichloroethane, trichloroethylene, perchloroethylene.

Anionic surfactants, such as linear alkylbenzenesulfonates, secondary alkanesulfonate, olefinsulfonate, alkyl sulfates, alkyl ether sulfates and alkyl ester sulfonates.

Further anionic surfactants are salts of acylaminocarboxylic acids, acyl sarcosinates, fatty acid-protein condensates, salts of alkylsulfamidocarboxylic acids, salts of alkyl and alkylaryl ether carboxylic acids, alkyl- and alkenylglyceryl sulfates, alkylphenol ether sulfates, alkyl phosphates, alkyl ether phosphates, isethionates, N-acyltaurides, alkyl succinates, sulfosuccinates, monoesters of the sulfosuccinates (particularly saturated and unsaturated C₁₂-C₁₈-monoesters) and diesters of the sulfosuccinates (particularly saturated and unsaturated C₁₂-C₁₈-diesters), acyl sarcosinates, sulfates of alkylpolysaccharides, such as sulfates of alkylpolyglycosides.

Nonionic surfactants are condensates of natural or synthetic, straight-chain or branched alcohols with about 1 to about 25 mol of ethylene oxide, mixed alkoxylates of these alcohols with ethylene oxide and propylene oxide or alcohol ethoxylates which are endcapped with an alkyl group, such as butyl; condensates of ethylene oxide having a hydrophobic basis, formed by condensation of propylene oxide with propylene glycol; condensates of ethylene oxide with a reaction product of propylene oxide and ethylenediamine; polyethylene, polypropylene and polybutylene oxide condensates of alkylphenols.

Further surfactants are alkyl- and alkenyloligoglycosides, fatty acid polyglycol esters, alkyloligoglycosides, alkenyloligoglycosides.

Nitrogen-containing components, such as amines (primary, secondary, tertiary amines), quaternary ammonium salts (preferably those which have no chloride as an opposite ion), fatty acid amides, di-, tri- and polyamines (e.g. alkylpropylenediamines, diethylenetriamine), aminoalcohols, polyaminoamides, amine oxides, fatty acid amides, such as coconut fatty acid diethanolamide, fatty amine polyglycol esters, fatty acid N-alkylglucamides, betaines, e.g. alkyldimethylammonium betaines, alkylamidobetaines, such as, for example, cocamidopropylbetaine, aminopropionates, aminoglycinates or amphoteric imidazolinium compounds, aminopropionates, aminoglycinates or amphoteric imidazolinium compounds.

Further substances which can be combined with the compound of the formula (1) for cleaning metal surfaces are alkali metal hydroxides; builders, such as carbonates (sodium carbonate), silicates, phosphates (alkali metal, ammonium and alkanolammonium salts of polyphosphates, such as, for example, sodium tripolyphosphate), borax and sodium carbonate; complexing agents, e.g. gluconates, phosphonates, such as ethane 1-hydroxy-1,1-diphosphonate, citric acid and its soluble salts, salts of polyacetic acids, such as, for example, ethylenediaminetetraacetic acid (EDTA) and nitrilotriacetic acid (NTA), polycarboxylates based on acrylic acid and maleic acid, copolymers of maleic anhydride with ethylene or vinyl methyl ether; soil release polymers, in particular soil release polyesters based on dicarboxylic acids and diols; foam enhancers, foam inhibitors, tarnish and/or corrosion inhibitors, emulsifiers, antioxidants and dispersants.

### EXAMPLES

For the investigations described below, tetramethoxyethane (TME) was chosen as an example of a solvent of the formula (1).

The following were used as references:

tetrachloroethene (=perchloroethylene=PER)
C₁₀₋₁₃-isoalkanes (=hydrocarbon solvents=HS)

### Example 1

### Cleaning a Metal Surface with Tetramethoxyethane to Remove Oil and Fat Dirt

The removal of liquid paraffin from metal parts with tetramethoxyethane was investigated in comparison with other solvents.

For this purpose, clean metal parts having dimensions of 2 cm×10 cm were dipped for 5 min at room temperature in liquid paraffin which was colored with the fat-soluble dye Sudan red. After excess oil had dripped off, the parts soiled in this manner were suspended in a beaker which was filled with tetramethoxyethane. This was stirred for 10 min at room temperature with a magnetic stirrer and the metal parts were then removed. These were then wiped with a paper tissue. The oily residue remaining on each metal part was determined visually from the staining of the paper tissue. For comparison, the experiment was repeated with tetrachloroethene and a C₁₀₋₁₃-isoalkane.

TABLE 1
Removal of liquid paraffin from metal parts by tetramethoxyethane
compared with PER and HS

| | Visual assessment of the | | |
|---|---|---|---|
| | paper tissue after cleaning | | |
| | the metal part with . . . | | |
| Metal part | tetramethoxyethane | PER | HS |
| Iron | clean | clean | clean |
| Stainless steel | clean | clean | clean |
| Copper | clean | clean | clean |

1. A method for cleaning metal parts in the industrial and commercial sector, wherein the metal parts are treated with a compound of the formula (1) 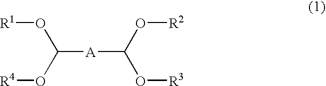
in which
A is (CH₂)ₐ or phenylene and R¹, R², R³ and R⁴ are identical or, independently of one another, are C₁ to C₆-n-alkyl or C₁ to C₆-isoalkyl or a mixture thereof and a is an integer from 0 to 4.

2. The method as claimed in claim 1, wherein the metal parts are treated with a compound of the formula (1), in which R¹, R², R³ and R⁴ are identical or, independently of one another, are C₁ to C₄-n-alkyl or C₁ to C₆-isoalkyl or a mixture thereof and A is a group of the formula (CH₂)ₐ and a is 0.

3. The method as claimed in claim 1, wherein the metal parts are treated with a compound of the formula (1) in which R¹, R², R³ and R⁴, independently of one another, are methyl or ethyl and A is a group of the formula (CH₂)ₐ and a is 0.

4. The method as claimed in claim 1, wherein the cleaning of the metal parts comprises treating said metal parts exclusively with a compound of the formula (1).

5. The method as claimed in claim 1, wherein the cleaning of the metal parts comprises treating said metal parts with a compound of the formula (1) and with the addition of other organic solvents.

6. The method as claimed in claim 1, wherein the cleaning of the metal parts comprises treating said metal parts with a compound of the formula (1) in a first treating step and treating said metal parts with other organic solvents in a separate cleaning step.

7. The method as claimed in claim 1, wherein the cleaning of the metal parts comprises treating said metal parts with a compound of the formula (1) with addition of up to 30% of water and with addition of up to 20% of an additional component selected from the group consisting of a surfactant, a builder, a complexing agent, a corrosion inhibitor and mixtures thereof.

8. The method as claimed in claim 1, wherein the cleaning of the metal parts comprises treating said metal parts in a first cleaning step with a compound of the formula (1), and then treating said metal parts with an aqueous aftertreatment.

9. The method as claimed in claim 1, wherein the cleaning of the metal parts comprises treating said metal parts in a first aqueous cleaning step, and then treating said metal parts in a second step with a compound of the formula (1).

10. The method as claimed in claim 1, wherein the compound of the formula (1) is used in combination with an additional component selected from the group consisting of anionic surfactants; nonionic surfactants; amphoteric surfactants; cationic surfactants; amines; amine derivatives; organic solvents; alkalis; builders; complexing agents; polymers copolymers of maleic anhydride with ethylene or vinyl methyl ether; soil release polymers; foam enhancers, foam inhibitors, tarnish and/or corrosion inhibitors, emulsifiers, antioxidants and dispersants.

11. The method as claimed in claim 1, wherein the cleaning of the metal parts comprises treating said metal parts with a compound of the formula (1) with addition of up to 20% of water and with addition of up to 10% of an additional component selected from the group consisting of a surfactant, a builder, a complexing agent, a corrosion inhibitor and mixtures thereof.

12. The method of claim 10, wherein the additional component is a polycarboxylates based on acrylic acid and maleic acid.

13. The method of claim 10, wherein the additional component is a soil release polyesters based on dicarboxylic acids and diols
